(19) United States
(12) Patent Application Publication
(10) Pub. No.: US 2009/0088604 A1
(43) Pub. Date: Apr. 2, 2009

(54) VERTEBRALLY-MOUNTED TISSUE RETRACTOR AND METHOD FOR USE IN SPINAL SURGERY

(76) Inventors: David Lowry, Holland, MI (US); Desmond O'Farrell, Grand Rapids, MI (US); Scott Tuinstra, Holland, MI (US); Roger Veldman, Hudsonville, MI (US); David Daugherty, Zeeland, MI (US); Gregory Fraley, Holland, MI (US); Kim Harrison, Holland, MI (US); Marlie Johnson, Holland, MI (US)

Correspondence Address: SHAY GLENN LLP, 2755 CAMPUS DRIVE, SUITE 210, SAN MATEO, CA, 94403, US

(21) Appl. No.: 12/239,431

(22) Filed: Sep. 26, 2008

Related U.S. Application Data
(60) Provisional application No. 60/976,331, filed on Sep. 28, 2007.

(51) Int. Cl. A61B 1/32

(52) U.S. Cl. 600/210; 600/231

(57) ABSTRACT

A retractor system and associated method are provided to manage soft tissue around a spinal surgical field. The system includes a hollow retractor with proximal and a distal apertures and an internal circumferential surface connecting the apertures, the surface and the apertures defining an operating volume and the area of the distal aperture defining an operating field. The system may further include other elements such as a handling tool to facilitate placement and removal of the retractor, a bone cutting tool, a trajectory control sleeve to guide the bone cutting tool, and/or an implantable bone plate upon which to position the retractor. A method for spinal surgery, particular from an anterior approach, includes positioning the retractor at a surgical site, and performing a medical procedure through the operating volume provided by the retractor.

### CROSS REFERENCE TO RELATED APPLICATIONS

This application claims priority to U.S. Provisional Patent Application No. 60/976,331 of Lowry et al., entitled “Vertebrally mounted tissue retractor and method for use in spinal surgery”, as filed on Sep. 28, 2007.

### FIELD OF THE INVENTION

The invention relates to devices and methods of spinal surgery. More particularly, the invention provides a device and method to achieve and maintain soft tissue retraction during spinal repair surgeries.

### INCORPORATION BY REFERENCE

All publications, patents and patent applications mentioned in this specification are herein incorporated by reference to the same extent as if each individual publication, patent or patent application was specifically and individually indicated to be incorporated by reference.

In particular, the following U.S. patent applications include related subject matter, and are incorporated in their entirety by this reference: U.S. patent application Ser. No. 11/855,124 of Lowry et al. (filed on Sep. 13, 2007, and entitled “Implantable bone plate system and related method for spinal repair”), U.S. patent application Ser. No. 12/210,109 of Lowry et al. (filed on Sep. 12, 2008, entitled “Device and method for tissue retraction in spinal surgery”), U.S. patent application Ser. No. 12/210,089 of Lowry et al. (filed on Sep. 12, 2008, entitled “Trans-corporeal spinal decompression and repair system and related method”, and U.S. Provisional Patent Application 60/990,587 of Lowry et al., as filed on Nov. 27, 2007, entitled “Methods and systems for repairing an intervertebral disk using a transcorporal approach”

### BACKGROUND OF THE INVENTION

The current standard treatment for neural decompression in the spine caused by a herniated disc, trauma, tumor, osteophyte or other compressing pathology is an anterior cervical discectomy and fusion (ACDF) or disc arthroplasty procedures, both of which entail removing the intervertebral disc, decompressing the neural element, and implanting a repair device between the adjacent vertebrae in the discal void. In this procedure, an incision is made in the anterior surface of the neck, followed by blunt finger dissection down to the spine. Handheld retractors are used to hold open the wound while multiple adjustable retractor blades are inserted within the wound, adjusted to expose the spine, and are then fixed in place. The disc tissue and neural compressing pathologies are removed, and a bone repair implant is placed in the disc space. When the repair device is a fusion implant, a cervical bone plate is then typically screwed onto the vertebrae above and below the implant to restrict motion and to positively locate the repair implant. The recovery time from this type of surgery is typically several months. During this time, the patient is subjected to physical limitations and is generally required to wear a neck support collar for at least some of the time period.

Retraction of surrounding soft tissues during the surgical procedure is necessary to prevent intrusion of tissue into the surgical field, which would impair surgical access and visibility, and to protect the tissue from contact with the surgical instruments. Currently available retractor systems (for example, U.S. Pat. No. 5,795,291 of Koros) can be subject to undesirable inter-operative shifting and migration within the wound. Such migration can require repositioning of the retractor during surgery; this is generally undesirable, but more specifically creates the risk of injury to adjacent soft tissue with each adjustment and prolongs the procedure.

Alternate surgical procedures to access spinal compressing pathologies been developed whereby the site of the compressing pathology is accessed through an adjacent vertebral body, thus reducing, or in some cases, eliminating the need for the removal of the complete inter-vertebral disc. Such procedures are described by Hong et al. (“Comparison between transuncal approach and upper vertebral transcorporeal approach for unilateral cervical radiculopathy—a preliminary report”, Minim Invasive Spine Surgery, 2006 October; 49 (5):296-301.) and by Jho et al. (“Anterior microforaminotomy for treatment of cervical radiculopathy: part 1: disc-preserving functional cervical disc surgery”, Neurosurgery 2002 November; 51, 5 Suppl.: S46-53), and include a discectomy by a transuncal approach, anterior microforaminotomy, and transcorporal approach procedures. These procedures provide for the removal of the offending pathology while preserving disc tissue, and they reduce or eliminate the need for a vertebral fusion or disc arthroplasty procedure.

These and similar alternative procedures are potentially less invasive than current procedures for having a smaller incision site, and an associated reduction in the risk of damage to adjacent tissue. Currently available surgical devices, however do not allow the full benefit of these methods to be realized. The current practice of using two pairs of opposing retractor blades requires an incision size that is larger than actually required by these procedures and exposes regions of the spinal column that are larger than necessary. Further, current retractor systems, in fact, do not satisfactorily protect surrounding nerve and vascular tissue. These retractor systems undesirably stretch surrounding tissues and the esophagus, causing with associated trauma, and, further, can pinch soft tissue between retractor blades with consequent bleeding and damage.

There is a need in the field for a minimally tissue retractor and management device applicable to spinal repair surgeries that positively engages the spine, isolates the surgical field from the adjacent soft tissue, and provides unrestricted operating and visual access to the surgical field. A desirable retractor system would protect the soft tissue adjacent to the surgical field, eliminate a need for inter-operative adjustment, and minimize wound size and the trauma that occurs while manipulating and controlling adjacent tissue during the surgical procedure.

### SUMMARY OF THE INVENTION

The invention provided herein includes embodiments of a retractor system configured to facilitate surgery on the spine, and methods with which to apply the device in order to facilitate surgical procedures. Embodiments of the system include a retractor that has a hollow or tubular structure with a proximal surface having a proximal aperture, a base comprising a distal aperture, the base configured to couple to one or more vertebral bodies, the distal aperture smaller than the proximal aperture; and an internal surface connecting the proximal aperture and the distal aperture, the surface and the apertures defining a distally-narrowing operating volume. The internal surface of retractor embodiments is typically circumferential, but some embodiments may have a partially-circumferential or circumferentially-interrupted internal surface. In some embodiments of the system, the base of the tubular structure includes a compressible member that is adapted to contact a vertebral body surface and form a seal around the aperture. The two-dimensional area defined by the boundary of the distal aperture may be appreciated as defining an operating field or window.

In some embodiments of the system, the retractor includes an engagement feature configured to engage a complementary feature of an insertion or handling tool that facilitates positioning and insertion of the retractor into a surgical site. In some embodiments of a method, the handling tool is further applied to facilitating removal the retractor from the surgical site. In some of these system embodiments, the engagement feature of the retractor is located on an internal surface of the hollow structure.

Some embodiments of the system include a trajectory control sleeve that is configured to be engageable to a retractor, and further, to accommodate at least a portion of a tissue-cutting or bone-cutting tool, such that the trajectory of the cutting tool (when the system is positioned in situ) is appropriately guided into an underlying surgical site. In some of these system embodiments, the trajectory control sleeve is configured to slidably engage within an internal aspect of the tubular structure. In some embodiments, a trajectory control sleeve engagement feature of the retractor is complementary to a retractor engagement feature of the trajectory control sleeve. And some embodiments of the system include a bone cutting tool, typically a rotary cutting tool, at least a portion of which is configured to be accommodatable by the trajectory control sleeve.

Some embodiments of the system include a handling tool that is adapted to facilitate positioning of the retractor at the surgical site preliminary to a procedure, and to facilitate removal of the retractor at the conclusion of a procedure. In some of these embodiments, the handling tool includes an engagement feature that is complementary to a handling tool-engagement feature of the retractor. And in some of these embodiments, the engagement feature of the handling tool is configured to compressibly-fit into a complementary engagement feature of the tubular structure.

Some embodiments of the system include one or more passages configured to accommodate fastening elements to attach the structure to a vertebral surgical site. Some embodiments of these passages for fastening elements include an abutting surface configured to limit distal movement of the fastening elements. Some embodiments of the fastening elements are integral with the retractor.

Some embodiments of the system include an implantable bone plate adapted to be engageable by the retractor. In some of these embodiments, the base of the retractor is adapted to engage the implantable bone plate. In some of these embodiments, the bone plate is configured to be attachable to one or more vertebral bodies.

In some embodiments of the system, the base of the retractor is configured and sized to fasten to a single vertebral body. In other embodiments of the system, the retractor is configured and sized to fasten to two adjacent vertebral bodies and to span an intervertebral space between the two bodies.

As noted above, embodiments of the invention also include methods with which to operate the retractor system summarized above in the context of performing a medical procedure on a spine from an anterior approach. Embodiments of the method include positioning a retractor device at a surgical site on the spine to retract soft tissue surrounding a surgical site (the retractor comprising a substantially tubular structure having a proximal aperture and a distal aperture); and performing the intended medical procedure, which may, for example, be an minimally interventional observation, a diagnostic procedure, an exploratory procedure, a therapeutic delivery procedure, or a surgical procedure. Typically, but not necessarily, the method further includes fastening the retractor with fastening elements to the surgical site, which may be localized to a single vertebral body, or may span two adjacent vertebral bodies and the intervening intervertebral space, or, in some instances may include spanning two or more vertebral bodies.

In some embodiments of the method, the positioning step includes positioning the retractor on an anterior aspect of a vertebral body of a cervical region of the spine. Typically, positioning occurs after a site has been exposed by an incision made by a conventional method. In other embodiments of the method, the positioning step includes positioning the retractor on an anterior aspect of a vertebral body in any of a thoracic, lumbar, or sacral region of a spine. And in some embodiments of the method, the positioning step includes positioning the retractor onto the surfaces of two adjacent vertebral bodies and spanning an intervertebral space between the two vertebral bodies.

In some embodiments of the method, after the step of disengaging and removing the handling tool, the method further includes engaging a trajectory control sleeve within the interior of the tubular structure of the retractor. Some of these embodiments further include engaging a cutting device within the trajectory control sleeve device. And some of these embodiments further include cutting vertebra; bone with the cutting device.

In some embodiments of the method, prior to the positioning step, the method includes engaging the retractor and a handling tool together. And in some of these embodiments, the positioning step includes positioning the retractor with the assistance of the handling tool.

Some embodiments of the method further include performing the surgical procedure in an operating field provided by an interior of the tubular structure of the retractor. Some embodiments of the method further include unfastening the retractor and removing it from the surgical site. In some of these embodiments, removing the retractor from the surgical site includes re-engaging the retractor and the handling tool, and removing the retractor from the surgical site with the assistance of the handling tool.

Some embodiments of the method include making use of an implantable bone plate. In such embodiments, for example, the positioning step may include securing a bone plate engageable by the retractor to the surgical site, and then securing the retractor to the bone plate.

### BRIEF DESCRIPTION OF THE DRAWINGS

FIG. 1 is a perspective view of a retractor device.

FIG. 2 is a longitudinal center-line sectional view of a retractor device.

FIG. 3 is a longitudinal cross sectional view of a retractor device with bone screws inserted.

FIG. 4 is an anterior plan view of a retractor device.

FIG. 5 is a perspective view of an alternate embodiment with vertebral engaging flaps.

FIG. 6 is a perspective view of an alternate embodiment with an engagement feature for a handling tool.

FIG. 7 is a cross section view of the device of FIG. 6 that shows an internal aspect of the engagement feature for a handling tool.

FIG. 8 shows a retractor device as installed spanning two adjacent vertebral bodies and spanning the intervertebral space between the two bodies.

FIG. 9 shows a trajectory control sleeve positioned relative to the retractor body.

FIGS. 10A and 10B show alternative embodiments of a retractor device.

FIG. 11 shows a retractor device engaging a bone plate of the type disclosed in U.S. patent application Ser. No. 11/855,124 of Lowry.

FIG. 12 a handling tool that is detachably engageable to a retractor device positioned to be inserted into a retractor.

FIG. 13 shows the mutual engagement of a handling tool and a retractor.

FIG. 14 shows a fastening pin that can be integrated into a retractor.

FIG. 15A is a perspective view of a retractor device with fastening pins of the type shown in FIG. 14 integrated into the device.

FIG. 15B is a cross section view of the retractor as shown in FIG. 15A with fastening pins integrated into the device.

### DETAILED DESCRIPTION OF THE INVENTION

The invention relates to device and methods for use in retracting soft tissue surrounding the spine or between the spine and the skin incision during spinal repair surgery. Embodiments of the retractor device include two major elements: (1) a vertebral mounting or contacting portion adapted on the base or distal aspect of the device that is adapted to substantially engage and conform to the anterior surface of one or more vertebral bodies, and (2) a solid walled hollow portion that generally stands at an angle that ranges between 45° and 90° relative to the vertebral mounting portion and whose accessible interior provides an operating volume and whose boundaries at a distal opening generally define an operating field. In some embodiments, the device is assembled as a device that includes integrated self-tapping screws that securely but detachably mount the retractor to the spine at a surgical site.

The hollow or tubular portion of the retractor is open at the proximal and distal ends, and the external wall of the tubular portion is typically (but not necessarily) uninterrupted and configured to prevent the ingress of retracted tissue into the surgical field. An operating space or volume is defined or circumscribed by the internal funnel-like aspect of the hollow portion. This hollow portion is generally funnel-shaped in that it has a proximal aperture generally larger than the distal aperture. This configuration for the device is generally advantageous for its intended use, which is to provide visibility for a surgeon, and to allow access of instruments from a relatively wide angle range that converges distally into a relative tight operating field. In some embodiments, the tubular portion is generally cylindrical or conical in shape, but these are not limiting shapes, as the device may also be rectilinear, and further, it is not necessarily symmetrical in its planar aspect or outline.

Further with regard to the hollow portion of the device that forms a generally funnel-shaped operating volume, the walls of the embodiments of the device are typically solid, and typically fully circumferential. However, some embodiments of the device, for particular applications, may have a wall that is not circumferentially complete. The wall may have, for example, describe a form that occupies an arc as narrow as 180°, or there may be embodiments that occupy an arc of 270°. Further, some embodiments of the device may be substantially fully circumferential, but include interrupting apertures or discontinuities in the wall.

The retractor device is securely fixed to the spine during the surgical procedure using fastening elements such self-tapping screws (or any other appropriate type of bone attachment or fastener element), which may be separate pieces or pieces integrated into or held within the retractor structure.

Inserting, positioning, and fastening of the retracting device within the wound may be assisted by the use of a handling tool that positively engages but easily and controllably detaches from a corresponding mating feature on the retractor device. This handling tool is detached from the device prior to performing the surgical procedure and can be re-engaged thereafter to assist in the removal of the retractor device prior to closure of the wound.

In some embodiments, a retracting device is sized to span two or more adjacent vertebrae and has fasteners inserted there through into at least one of the vertebral bodies. In some embodiments, the device may be mounted onto a single vertebra and affixed there by at least one fastener.

In some embodiments, the retractor device may detachably engage a bone plate device such as that disclosed in U.S. patent application Ser. No. 11/855,124 of Lowry et al. (entitled “Implantable bone plate system and related method for spinal repair”, as filed on Sep. 13, 2007), which has already been attached to one or more vertebrae.

According to an aspect of the invention, the main tubular body portion of the device is formed of a substantially rigid biocompatible material. The contact surfaces for engaging vertebral bone tissue may be formed from a different biocompatible material, different by being compressible. The compressible feature of the composition provides conformability to the vertebral surface, and a sealability that substantially prevents entry of tissue or fluids into the surgical field. The generally rigid biocompatible materials may be polymeric, metallic, ceramic, or a combination thereof; the compressible or conformable biocompatible materials are more generally polymeric or elastomeric, or have a high proportion of polymer in their composition.

In another aspect of the invention, a method is provided for inserting the soft tissue retractor through an incision, attaching the retractor to one or more vertebrae, and performing a surgical repair procedure through the retractor. Embodiments of the method typically start by creating of an incision in the surface of the skin and manually retracting the tissue to expose the spine by standard procedures. Inventive aspects of embodiments of the method include positioning the retractor at the site with a handling tool, securing the retractor to the surgical site with one or more fasteners, disengaging and removing the handling tool, and performing the intended surgical procedure. Embodiments of the method may optionally further include re-engaging the retractor and the handling tool, unfastening the retractor from the bone, removing the retractor device from the surgical site, and then closing the incision. In some embodiments of the method, the retractor is positioned so as to span two adjacent vertebrae, thereby exposing intervertebral disc and vertebral bone tissue to the surgeon. In other embodiments the retraction device is placed on a single vertebral body so as to expose anterior or lateral vertebral bone tissue only.

According to some aspects of the invention, methods are provided for establishing and controlling the trajectory of a tissue-cutting device, more particularly, a bone cutting device such as a drill, burr, or reamer, by the inserting and engaging a trajectory control device within the internal volume of the retractor after it has been affixed to the vertebral bone tissue. The trajectory control sleeve has an orientation feature that positively engages a corresponding feature on the retractor device so as to control the orientation of the trajectory control sleeve axes relative to the retractor and vertebral bodies. A detailed disclosure of embodiments of another embodiment of a trajectory control sleeve and methods for use therefore are provided in U.S. patent application Ser. No. 12/210,192 of Lowery et al., (filed Sep. 12, 2008), which is incorporated herein in its entirety.

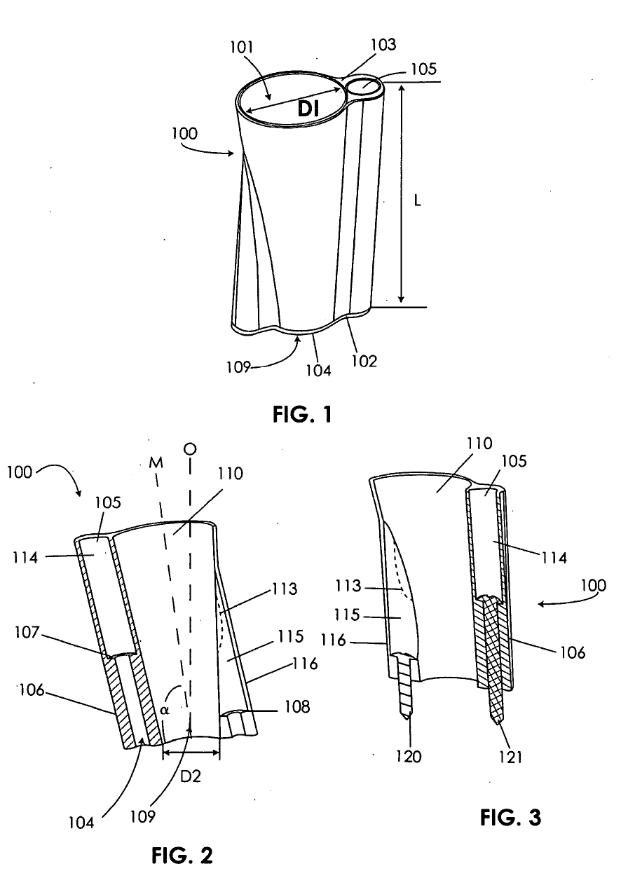

Some of these embodiments and features of the present invention as described above are depicted as examples in FIGS. 1-13, and will now be detailed.

FIGS. 1-4 show embodiments of a retractor device 100 with a proximal surface 103 and a distal surface 104, the distal surface being adapted to generally engage the anterior surface of vertebral bone upon insertion within an incised wound. Each surface (proximal and distal) includes a rim that defines, respectively, an anterior aperture 101 and a distal aperture 109. Inasmuch as the retractor is configured to generally approach a vertebral surgical site from the anterior aspect of the spine, from the perspective of an operating surgeon, the distal aspect or surface of the retractor makes contact with the anatomically anterior aspect of the spine, and the proximal aspect of the retractor includes an aperture that provides surgical access to a working volume and surgical field within the hollow confines of the retractor. The embodiment shown in FIG. 1 includes a compressible polymeric member 102 on the distal surface 104 that provides a conformable surface adapted to form a seal between the distal surface of the retractor device and the anterior surface of a vertebral body against which it is compressed. This sealing element 102 further provides a degree of vibration dampening so as to minimize the risk of loosening of the retractor device 100 during the procedure.

The embodiments of device 100, as shown in FIG. 1, have an overall length L that typically may vary within range of about 35 mm to about 90 mm. An anterior access aperture 101 has a diameter D in the range of about 10 mm to about 35 mm. These dimensions for diameter and length are typical but merely exemplary, and the scope of invention includes devices of the general form described but with smaller or larger dimensions. The appropriate length L and diameter D1 of a device 100 may vary according to the specifics of the anatomy of the patient and the surgical site, and according to the discretion of the operating surgeon. Diameter D2, in particular, is determined by the minimum access requirements of the procedure to be performed and is selected by the surgeon.

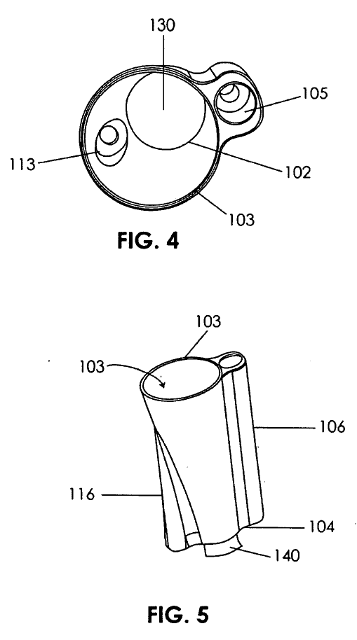

FIGS. 2 and 3 provide an axial centerline section views through the device 100. The device 100 provides an open surgical operating volume 130 (FIG. 4) in the form of a space surrounded by interior surface or wall 110 and between the anterior access aperture 101 and the posterior vertebral bone access aperture 109, through which the medical procedure may be performed.

FIG. 2 also shows a line O that serves as a reference for a line that originates at the center of distal aperture 109 and is positioned orthogonal to the base surface 104 of retractor 100. Line M marks a line that also originates at the center of distal aperture but is positioned at the central axis of the funnel-shaped aspect of retractor 100. The angle of the line M with respect to the base 104 may is labeled as angle α, and it may vary in various embodiments of the retractor, as discussed further below. In some embodiments, those, for example, that have a funnel portion between proximal and distal orifices that is flattened or lengthened in comparison to circular profile, such as an oval or rectilinear form, the center of the funnel-space may be better described with a central plane rather than a central axial line, however, an angle α also characterizes the angular disposition of the funnel-shaped interior aspect of the device with reference to its base. In some embodiments of the retractor, angle α may, in fact, have the maximal value of 90° (orthogonal to the base). In other embodiments, angle α may be acute, and range to a value as low as about 45°, merely by way of example. The angle α of the device desirably aligns with the approach being taken by an operating surgeon. and can also be adjusted by varying the configuration of the base of the retractor, as it may, for example, assume a wedge-shape, with one side raised or thicker than the opposite side.

Referring to FIGS. 2-4, the device may be affixed to vertebral bone by the insertion of one or more fastening elements such as bone screws through the anterior facing openings 105 and 113; thereafter the screws pass through passages 114 and 115 within the retractor device and screwably engage sites on the anterior aspect of the vertebral bone surface. Abutting surfaces 107 and 108 are adapted to restrict the distal travel of the bone screw so as to prevent over penetration into the vertebral body, while also being adapted to assure a compressive engagement of the retractor against the vertebral body. FIG. 3 shows bone screws 120 and 121 in their final position after insertion through the device into vertebral bone tissue.

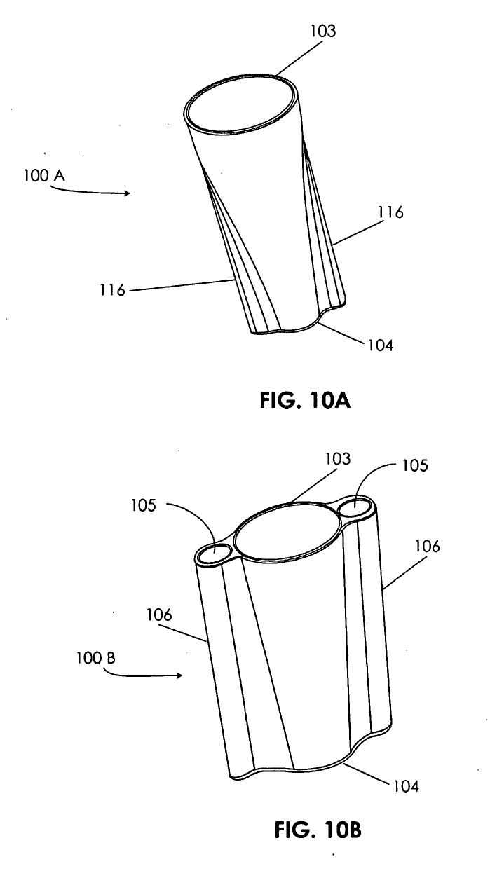

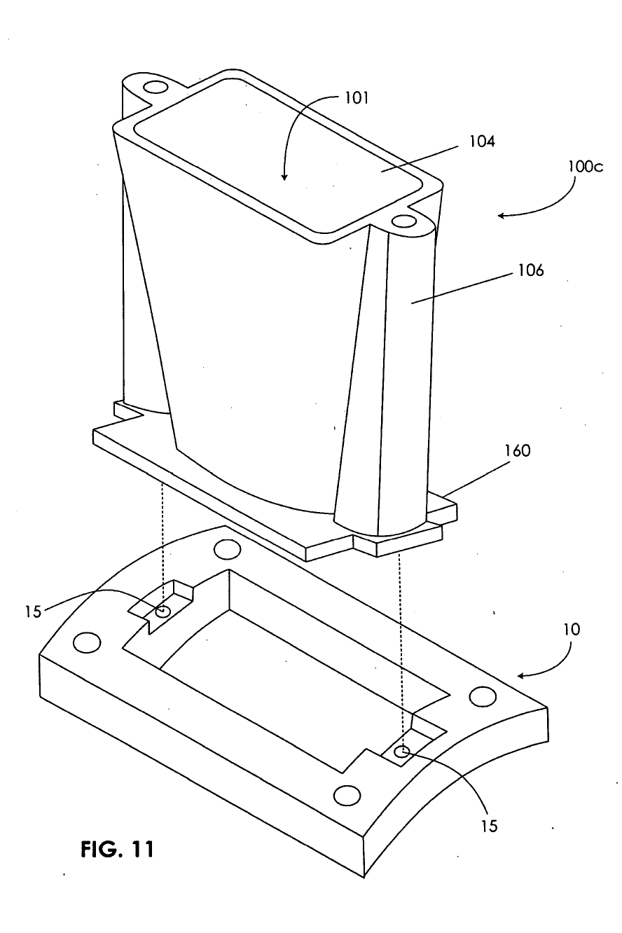

It can be appreciated from the embodiment depicted in FIGS. 1-4 that there are at least two alternative configurations for channels that fastening elements. The channel 115, for example, has a proximal opening 113 that is within the funnel-shaped internal aspect of device 100, and that channel emerges distally from the confines of the funnel in an externally manifesting fin-like structure 116. As an alternative form, channel 114, with proximal opening 105 outside of the confines of the funnel-shaped internal aspect of device 100, manifests externally as a silo-like structure 106. The particular embodiments of FIG. 1-4 are examples, thus, in which one fastening element passage originates within the funnel-shaped portion of the device, and the other passage way is wholly external to the funnel-shaped portion of the device. Other exemplary embodiments are noted below, and depicted in FIGS. 10A and 10B.

Referring again to FIGS. 1 and 4, after the device 100 is affixed to vertebral bone tissue by way of screws 120 and 121, manual retractor instruments that have been used to facilitate its positioning are removed from the wound, and the soft tissue adjacent the wound is permitted to relax and to compressively engage the external surfaces of the device. The combination of (1) the positive fixation of device 100 to the vertebral bone and (2) the uniform circumferential compressive forces of the retracted soft tissue in contact with the exterior surfaces of the device 100 together provides substantial mechanical stability and resilience to the seated device and restricts entry of soft tissue into the surgical field within the retractor. After fixation to the vertebral bone tissue, the surgical field is accessed through the operating volume 130 with instruments inserted through the anterior facing aperture 101 and by engagement of the vertebral tissue through the posterior aperture 109.

In brief, with regard to embodiments of retractor 100 as seen in FIGS. 1-4, opening 105 provides access to passage 114 which appears as a silo or side post 106 to the hollow or funnel-shaped portion of the retractor; and passage 114 accommodates screw 121, whose forward movement is contained by abutment 107. On the opposite side of the retractor, opening 113, within the internal aspect and operating volume 130 of the device, provides access to passage 115; and passage 115 accommodates screw 122, whose forward movement is contained by abutment 108.

As a consequence of the surgical working field being exclusively within the enclosed volume 130 of the device 100, there is no contact between surgical instruments and adjacent tissue, thus substantially eliminating risk of damage to the tissue. On completion of the surgical procedure, the fixing screws 120 and 121 are accessed through the anterior facing holes 113 and 105, respectively, and withdrawn from their vertebral body location, and device 100 is withdrawn from the surgical site.

FIG. 5 shows an alternate embodiment of the device with an integral flexible member 140 at the distal end of device 100. Member 140 is adapted to flexibly engage vertebral bone that it contacts, thereby forming a barrier seal that prevents or restricts ingress of soft tissue between the device and the vertebral body after the removal of the manual retractor devices.

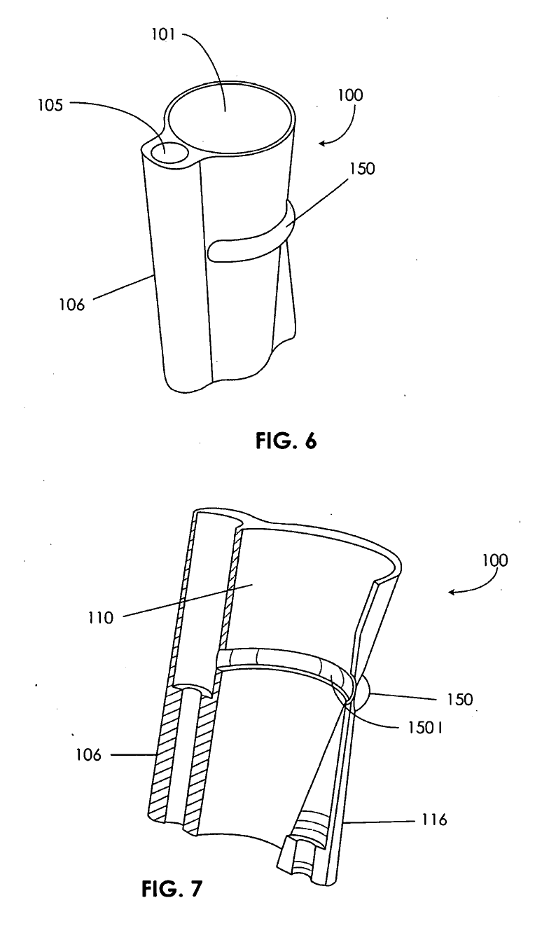

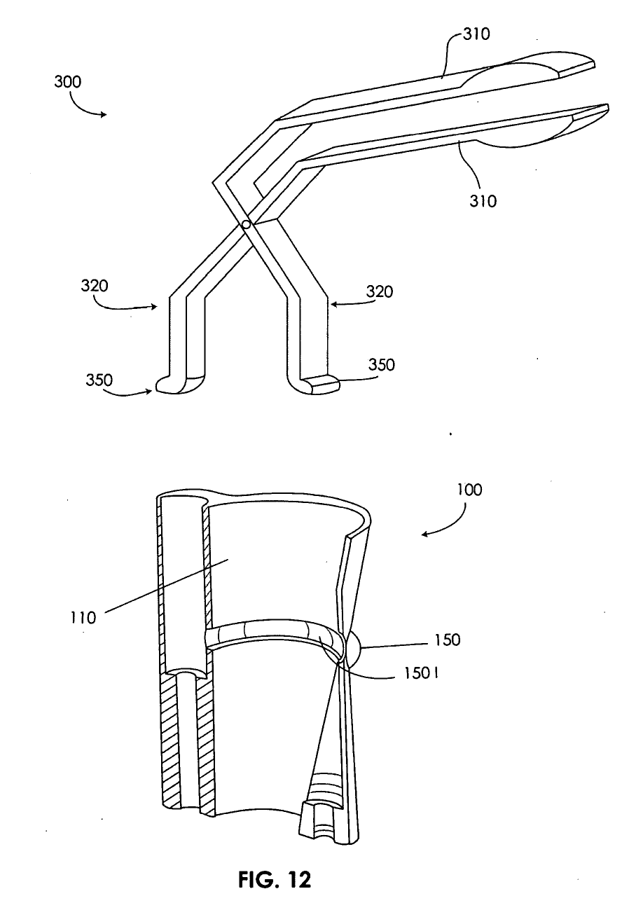

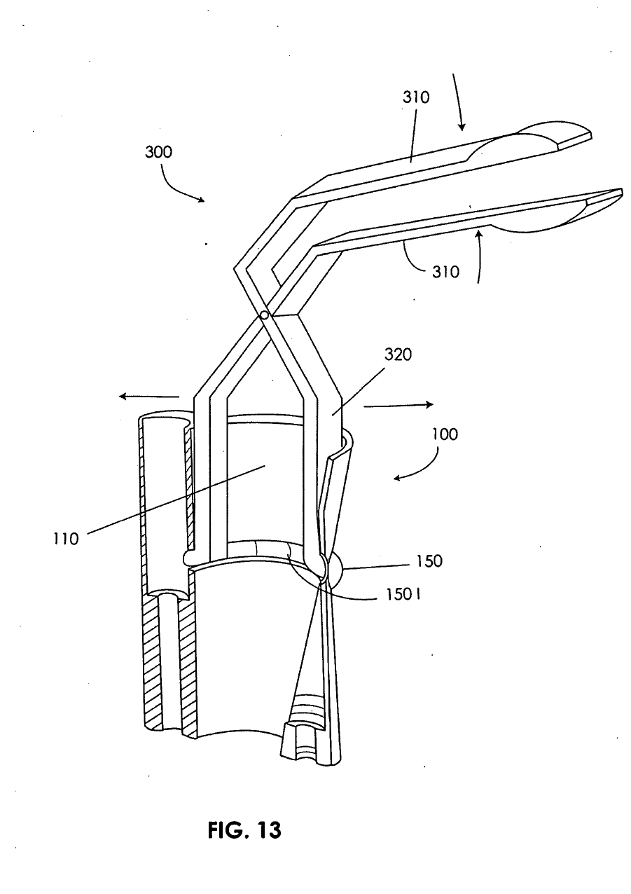

FIGS. 6 and 7 show external perspective and internal views, respectively of an embodiment of a retractor 100 with an engagement feature 150 for positively locating and engaging a handling tool within the retractor. Engagement feature 150 manifests externally as a raised annular form and manifests internally as a slot 150I. In some embodiments, the internal surface or wall 110 of retractor 100 may be sufficiently thick that slot 150I can be accommodated within the thickness of wall such that an external annular feature is not necessary. In this embodiment, one or more corresponding features on a handling tool (FIGS. 12 and 13) are lockably-engageable to an internal aspect 150I of the engagement feature, and more generally compressively engage the internal surface or wall 110 of retractor 100.

FIG. 12 shows a handling tool 300 positioned as if about to engage a retractor 100; FIG. 13 shows a handling tool 300 and a retractor 100 engaged such that the handling tool can facilitate handling and placement of the retractor into position on a vertebral site. The handling tool includes radially expansive elements 320 that can be manipulated with manual elements or handles 310. A retractor-engaging feature 350 is located on a radially-outward facing surface of each expansive element 320 of handling tool 300. By inserting the radially expansive elements of handling tool 300 into the interior of the retractor and providing radially expansive force, the retractor engaging feature 350 and positively engage the handling tool engagement feature or slot 150I of retractor embodiment 100. The handling device embodiment 300 shown, and its expansive elements 320 and engagement features 350 are merely examples of many alternative embodiments that would operate in a similar manner, as can be appreciated by those in the art, all of which are included as embodiments of the invention. A feature common to these embodiments is that the retractor-engaging element of a handling tool and the handling tool engagement feature of a retractor are complementary to each other. The engagement interaction is typically under manual control by a surgeon. In some embodiments, a temporary locking mechanism is included in the engagement site, or elsewhere in handling tool 300, such that the handling tool can hold the retractor without continuous force being provided by the surgeon.

After grasping a retractor 100 with a handling tool 300, a surgeon can position the retractor into a surgical site that has been exposed on an anterior aspect of the spine. With insertion into the wound, the compressive locking force is released and the handling tool is removed from the device. In some embodiments of a method, particularly when the embodiment of the retractor 100 includes a fastening element passageway 105 within silo-like structure 106, the surgeon may continue to hold the retractor in position with handling tool 300 while securing the device to bone at the surgical site. At some point, however, either before or after completing the fastening of the retractor to the surgical site, the compressive engagement of the handling tool within the retractor is released, and the handling tool is withdrawn. With the retractor in place, and the operating field clear of impinging soft tissue by virtue of its retraction by the retractor, the surgical procedure is conducted. On completion of the surgical procedure, the retractor is removed from the site; in some embodiments of a method, handling tool 300 may be re-inserted and lockably re-engaged with interior surfaces of the device 100 to assist in screw back-out and device removal.

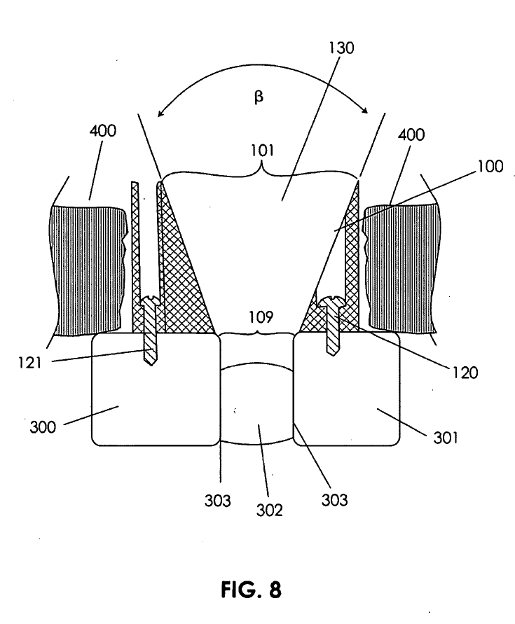

FIG. 8 shows a cross-sectional view of device 100 in an installed or in situ position spanning two adjacent vertebrae 300 and 301, and being affixed there by the mounting screws 120 and 121. Retracted soft tissue 400 overlaying the vertebral bodies compressively engages the external aspect of the device 100, such engagement preventing ingress of the tissue into an operating volume and the surgical field as exposed through the vertebral access aperture 109. The device may be positioned on the vertebral bodies 301 and 302 to expose vertebral bone tissue and/or intervertebral disc tissue 302. Further shown in FIG. 8 is the cleared operating volume 130 provided within the confines of the hollow, funnel-shaped portion of the device, as well as the range of motion available for manipulating the surgical instruments within the device, described by the angle β. FIG. 8 shows a device 100 implanted across two adjacent vertebral bodies, however in other embodiments of methods of applying the device 100, it may be positioned on a single vertebral body, and used to create an operating volume and surgical field that is wholly centered on that single vertebral body.

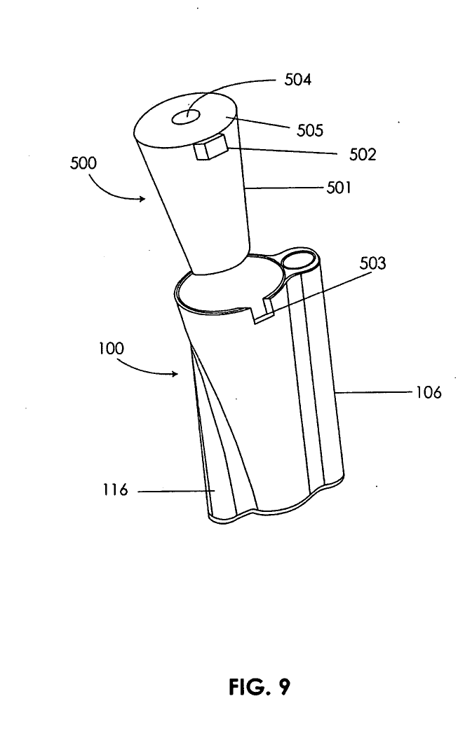

FIG. 9 shows a trajectory control sleeve insert 500 in relative position to a retractor device 100, into which it can be inserted temporarily in order to guide a bone cutting tool (not shown). Embodiments of the trajectory control sleeve 500 typically have an external surface geometry that is substantially complimentary to the internal geometry of retractor device 100, and the sleeve as a whole is adapted to be slidably-engageable within the retractor. Embodiments of the trajectory control sleeve have an engaging member or feature 502 that has a defined spatial relationship to the axes of the trajectory control sleeve channel 504 in order to establish and control the point of entry and penetration of a tissue cutting tool, typically a rotary cutting tool, into bone.

Trajectory control sleeve 500 further has a proximal abutting surface 505 adapted to engage a bone-cutting tool (as it is seated and operating within the sleeve) in order to control the depth of cutting tool penetration through the device and into the bone tissue below. The engaging member 502 positively engages a corresponding feature 503 on the retractor device 100. Engagement of the feature 502 of the trajectory control sleeve insert and feature 503 of the retractor 100 prevents unwanted movement of the sleeve during bone cutting and assures a well-controlled trajectory of the drill into the underlying bone tissue. The trajectory control sleeve 500 can be further or alternatively engaged with the retractor device 100 by various locking-but-releasable mechanisms, such as snap and bayonet mechanisms, so as to assure positive and orientation-specific engagement of the elements.

The trajectory channel 504, as shown in FIG. 9, is centered in within proximal surface 505, and though not shown, may be assumed to penetrate proximally at an angle orthogonal to surface 505. Alternative embodiments, however, may be configured such that the channel 504 is located at a site other than the center of surface 504, and penetrates distally at a non-orthogonal angle. Thus, by a combination of control of variable angles α of the funnel-shaped interior of device 100 as whole (as described in the context of FIG. 2), and variable orientations of the trajectory channel 504, the trajectory of a rotary cutting tool can be very precisely controlled to achieve a desired trajectory. Depending on the surgical site at which a retractor is implanted and the desired procedure, a rotary cutting tool may be directed into vertebral bone or into disc tissue in an intervertebral space.

Exemplary device 100 embodiments described and depicted (FIGS. 1-9) to this point have generally circular or ovular apertures (proximal and distal) and internal funnel-like aspects that are substantially cylindrical in form. It should be appreciated, however, that embodiments of the device can have alternative shapes, such as, for example, various rectilinear forms (see FIG. 11), as may be appropriate or provide particular advantages for a specified procedure, pathology, or surgical site. Further, these embodiments provide an operating volume 130, as bounded by internal surface of wall 110, which is configured such that a central axis midline M (see FIG. 2) forms a non-orthogonal angle with respect to the base 104 of the retractor. This angled-funnel aspect of the retractor can be advantageous in that a surgeon typically operates from a positionally-biased perspective, and an optimal use of a retracted volume or operating field can be one wherein the cleared operating volume is centered with respect to the surgeon's perspective and angle of approach. In alternative embodiments, however, the operating volume as bounded by an internal can be oriented such that it is centered about a midline that is orthogonal to the base.

FIGS. 10A and 10B show embodiments of a retractor that provide alternatives to those depicted in FIGS. 1-9. FIG. 10A shows an embodiment of a device 100A that includes two passageways for fastening elements that originate proximally as openings within the funnel-shaped internal aspect of the device, and manifest outwardly as fin-like structures 116. FIG. 10B shows an embodiment of a device 100B that includes two passageways for fastening elements that originate as proximal openings 105 and extend to the distal surface 104 in the form of an outwardly manifesting silo-like structure 106.

FIG. 11 shows a retractor device 100C engaging an implantable bone plate 10 of the type disclosed in U.S. patent application Ser. No. 11/855,124 of Lowry et al. (filed Sep. 13, 2007) and in U.S. patent application Ser. No. 12/210,089 of Lowry et al. (filed Sep. 12, 2008), both of which are incorporated into this application in their entirety. This particular exemplary retractor embodiment 100C includes fastening element passageways that manifest as external silo-shaped structures 106 that distally terminate on a base plate 160. Base plate 160 and bone plate embodiment 10 are configured to be mutually engageable and securely-connectable by fastening elements (not shown) that extend from passageways 114 and into openings 15 of bone plate 10. This retractor embodiment 100C also provides an example of an alternative shape (compared to the generally circular profile and cylindrical form depicted in FIGS. 1-9) which is extended into a rectilinear form. Device 100C, when mounted to an implantable bone plate 10, serves a surgical site in a same manner similar to that of the retractor system disclosed in U.S. patent application Ser. No. 12/210,089, and as such, its dimensions may appropriate to permit spanning more than two vertebral bodies.

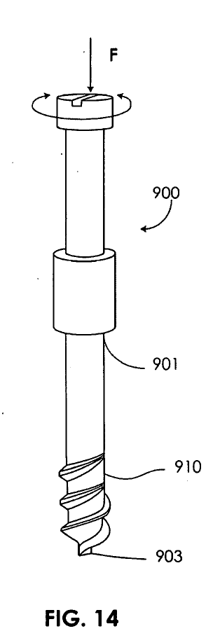

FIG. 14 shows an embodiment of a fastening pin 900 that can be integrated into a retractor. Integration of fastening pins into the device can be advantageous during a surgical procedure simply by removing the complication of handling loose screws, and for maintaining sterility of components. Fastening pin 900 includes a keeper feature 901 formed of a pliable or compliant material that compressible fits within a passage of the device and holds it with sufficient force that is retained within the passage under normal handling conditions. Accordingly, the keeper element 901 is placed along the length of the pin 900 at a calibrated position. The force holding the keeper feature 901 within the passage, however, is sufficiently weak that it can easily be overcome by manually applied force F that is directed distally such that the pin is driven through the passage and into underlying vertebral bone, and is also sufficiently weak that it can easily be overcome by manually applied force pulling the pin proximally, as in an extraction procedure. The distal-facing surface of the keeper feature 901 also represents an abutting surface that prevents distal movement of the fastening pin beyond a prudent limit. The pin 900 has a head 902 that is adapted to receive rotational force that is translatable into distally-driven force by a threaded portion 910. The head 902 may additionally have features (not shown) that facilitate counter rotation or gripping as occurs when the pin is being extracted from vertebral bone, in preparation for removing a retractor. Pin 900 also has a self-tapping, penetrating, or cutting surface 903 at its distal end that is adapted to enter a bone surface and penetrate into bone tissue as the pin is rotated.

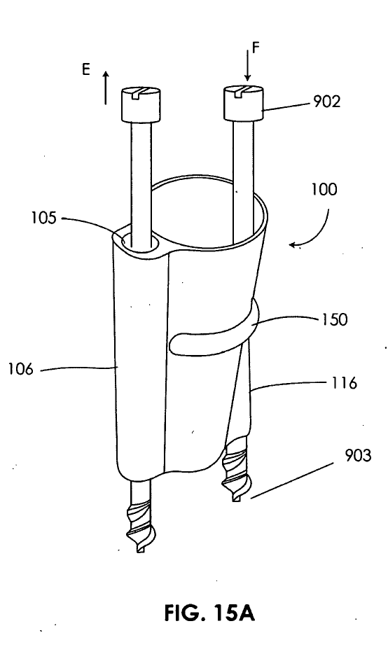

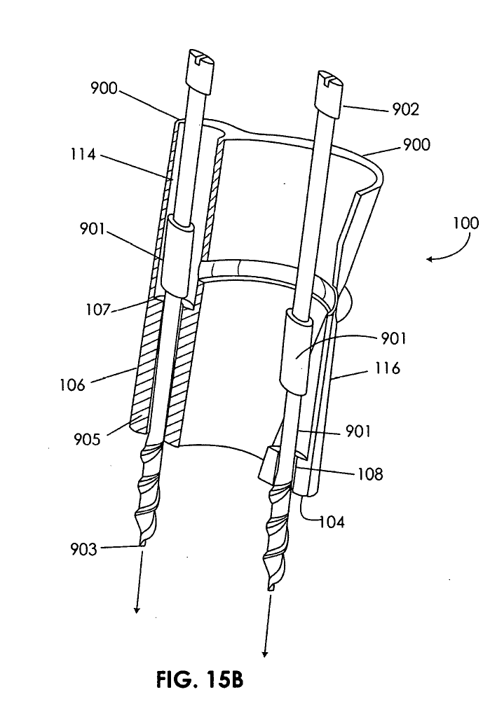

FIG. 15A is a perspective view of a retractor device with device-integrated fastening pins of the type shown in FIG. 14 integrated into the device. The embodiment and the perspective with which it is depicted are similar to the embodiment shown in FIG. 6. Visible above the proximal surface of the device 100 are the upper portions of two pins 900 and their heads 902, a pin emerging from each of the two openings for fastening element passageways. Opening 105 is visible in this view, opening 113 is within the funnel-shaped portion of the device and not visible in this view.

FIG. 15B is a cross section view of the retractor as shown in FIG. 15A with fastening pins integrated into the device, the view as a whole being similar to that of FIG. 7. In this view, the keeper features 901 of pins 900 are each visible in their respective passage ways 114 and 115. Shown in this view is abutting surface 107 that is adapted to engage a fastening element 900 and limit its distal movement within passageway 114, as well as abutting surface 108 that is adapted to engage a fastening element 900 and limits its distal movement within passageway 115. Cutting or penetrating surfaces 903 at the distal end of pins 900 can be seen extending beyond the distal base 104 of the retractor.

While the description provided herein of devices and methods by which to operate the devices refer in an exemplary manner to procedures applied to the cervical spine by way of an anterior approach, such devices and methods may also be applicable to other neurosurgical and orthopedic procedures. Surgeries in the thoracic, lumbar, and sacral regions of the spine, with anterior, posterior, or lateral approaches, also require the retraction of surrounding soft tissue structures, such as the pleura and its contents, the peritoneum and its contents, adjacent musculature, and/or adjacent vascular structures. For procedures in these regions, there is also no currently available retractor system that attaches directly to the spine and there stabilizes a hollow structure to enable the performance of a repair procedure there through, to protect adjacent tissue from instrument injury, or to facilitate illumination and visualization of the surgical field during a procedure.

What is claimed is:

1. A retractor system for a surgical site on a spine comprising a retractor, the retractor being a hollow structure comprising:
a proximal surface having a proximal aperture,
a base comprising a distal aperture, the base configured to couple to one or more vertebral bodies, the distal aperture smaller than the proximal aperture; and
an internal surface connecting the proximal aperture and the distal aperture, the surface and the apertures defining a distally-narrowing operating volume.

2. The retractor system of claim 1 wherein the base comprises a compressible member adapted to contact a vertebral body surface and form a seal around the aperture.

3. The retractor system of claim 1 wherein the proximal aperture is larger than the distal aperture such that the operating volume narrows from the proximal aperture to the distal aperture.

4. The retractor system of claim 1 wherein internal surface connecting the proximal aperture and the distal aperture is fully circumferential around the operating volume.

5. The retractor system of claim 1 wherein the retractor comprises an engagement feature configured to engage a complementary feature of a handling tool.

6. The retractor system of claim 5 wherein the engagement feature of the retractor is located on an internal surface of the tubular structure.

7. The retractor system of claim 1 further comprising a trajectory control sleeve configured to be engageable to the retractor and configured to receive at least a portion of tissue cutting tool.

8. The retractor system of claim 7 wherein the trajectory control sleeve is configured to slidably engage within an internal aspect of the tubular structure.

9. The retractor system of claim 1 wherein the retractor comprises a trajectory control sleeve engagement feature that is complementary to a retractor engagement feature of a trajectory control sleeve.

10. The retractor system of claim 1 further comprising a handling tool adapted to facilitate positioning of the retractor at the surgical site.

11. The retractor system of claim 10 wherein the handling tool comprises an engagement feature complementary to a handling tool-engagement feature of the retractor.

12. The retractor system of claim 11 wherein the engagement feature of the handling tool is configured to compressibly fit into a complementary engagement feature of the tubular structure.

13. The retractor system of claim 1 further comprising a tissue cutting tool, at least a portion of which may be accommodated by a trajectory control sleeve that configured to be engageable to the retractor and configured to receive at least a portion of rotary cutting tool.

14. The retractor system of claim 1 wherein the retractor comprises one or more passages configured to accommodate fastening elements to attach the structure to a vertebral surgical site.

15. The retractor system of claim 14 wherein the one or more passages for fastening elements comprise an abutting surface configured to limit distal movement of the fastening elements.

16. The retractor of claim 14 wherein the fastening elements are integral with the retractor.

17. The retractor system of claim 1 further comprising an implantable bone plate adapted to be engageable by the retractor.

18. The retractor system of claim 17 wherein the base of the retractor is adapted to engage the implantable bone plate.

19. The retractor system of claim 17 wherein the bone plate is configured to be attachable to one or more vertebral bodies.

20. The retractor system of claim 1 wherein the base of the retractor is configured and sized to fasten to a single vertebral body.

21. The retractor system of claim 1 wherein the base of the retractor is configured and sized to fasten to two adjacent vertebral bodies and to span an intervertebral space between the two bodies.

22. A method for performing a medical procedure on a spine from an anterior approach comprising:
positioning a retractor at a surgical site on the spine such that the retractor contacts one or more vertebral bodies and maintains retraction of soft tissue surrounding the site, the retractor being a hollow structure comprising a proximal surface having a proximal aperture, a base comprising a distal aperture, the base configured to couple to one or more vertebral bodies, the distal aperture smaller than the proximal aperture; and an internal surface connecting the proximal aperture and the distal aperture, the surface and the apertures defining a distally-narrowing operating volume; and
performing the medical procedure through the operating volume of the retractor.

23. The method of claim 22 further comprising fastening the retractor to one or more vertebral bodies.

24. The method of claim 22 wherein the positioning step comprises positioning the retractor on an anterior aspect of a vertebral body of a cervical region of the spine.

25. The method of claim 22 wherein the positioning step comprises positioning the retractor on an anterior aspect of a vertebral body in any of a thoracic, lumbar, or sacral region of a spine.

26. The method of claim 22 wherein the positioning step comprises positioning the retractor onto the surfaces of two adjacent vertebral bodies and spanning an intervertebral space between the two vertebral bodies.

27. The method of claim 22, after the positioning step, further comprising engaging a trajectory control sleeve within the interior of the tubular structure of the retractor, the trajectory control sleeve configured to be engageable to the retractor and configured to receive at least a portion of tissue cutting tool.

28. The method of claim 27, further comprising engaging a tissue-cutting device within the trajectory control sleeve device.

29. The method of claim 28, further comprising cutting bone with the tissue-cutting device.

30. The method of claim 22, prior to the positioning step, comprising engaging the retractor and a handling tool together.

31. The method of claim 30 wherein the positioning step comprises positioning the retractor with the assistance of the handling tool.

32. The method of claim 23 further comprising unfastening the retractor and removing it from the surgical site.

33. The method of claim 32 wherein removing the retractor from the surgical site comprises engaging a handling tool to the retractor, and removing the retractor from the surgical site with the assistance of the handling tool.

34. The method of claim 22, wherein the positioning step comprises securing a bone plate engageable by the retractor to the surgical site, and securing the retractor to the bone plate.
